(19) United States
(12) Patent Application Publication
(10) Pub. No.: US 2011/0144629 A1
(43) Pub. Date: Jun. 16, 2011

(54) Method for Complementing Conventional Vision Correction with Laser Correction of the Cornea

(76) Inventors: Rupert Veith, Pfarrkirchen (DE); Kristian Hohla, Munchen (DE); Loesel Frieder, Mannheim (DE)

(21) Appl. No.: 12/961,363

(22) Filed: Dec. 6, 2010

Related U.S. Application Data
(60) Provisional application No. 61/285,373, filed on Dec. 10, 2009.

(51) Int. Cl. A61F 9/008; A61B 3/00

(52) U.S. Cl. 606/5; 351/246

(57) ABSTRACT

A system and method for reshaping the cornea of an eye with a laser that also incorporates a subsequent compensation for residual vision aberrations. A diagnostic unit obtains a pre-op prescription for the eye and predicts an induced refractive shift resulting from laser treatment. Based on this data, a preview lens is selected or manufactured to evaluate the tolerance of a patient to a predicted post-op vision. Then, a laser unit is used to alter the cornea of the eye (i.e. laser treatment) to increase the depth-of-field of the eye. Subsequently, after the laser treatment has been completed, conventional vision correction is provided by spectacles, intraocular lenses or contact lenses. This conventional correction will then compensate for any remaining pre-op aberrations, as well as any residual aberrations in the patient's post-op vision that may have been induced during the laser treatment.

The application claims the benefit of U.S. Provisional Patent Application Ser. No. 61/285,373, filed Dec. 10, 2009.

### FIELD OF THE INVENTION

The present invention pertains generally to systems and methods for reshaping and structurally altering the cornea of an eye to improve the vision of a patient. More particularly, the present invention pertains to systems and methods that combine intrastromal laser treatment with conventional vision correction to improve the vision of a patient. The present invention is particularly, but not exclusively, useful as a system and method for reshaping the cornea of an eye, wherein an intrastromal laser corrects the depth-of-field of the eye, and wherein a conventional vision correction is used to compensate for any pre-op or post-op aberrations.

### BACKGROUND OF THE INVENTION

Recently, laser surgical procedures have become a common way to treat various diseases of the eye. Presbyopia is one such disease. A patient suffering from presbyopia has a diminished ability to focus on near objects because of a decreased accommodation of the lens of the eye. Simple solutions, such as over-the-counter reading glasses, may help a patient overcome some of the symptoms associated with presbyopia. But, in many cases, a laser procedure that alters the cornea, thus increasing depth-of-field of the eye, is a more appropriate treatment option for presbyopia. In addition, a patient with presbyopia may also be near-sighted or far-sighted. Thus, a laser treatment used to treat presbyopia, or any other health condition associated with the eye, may also need to account for more than one disorder of the eye to yield the best possible results for an individual patient.

In many laser procedures, it is desirable to modify the optical performance of the cornea. The cornea of an eye has five (5) different identifiable layers of tissue. Proceeding in a posterior direction from the anterior surface of the cornea, these layers are: the epithelium; Bowman's capsule (membrane); the stroma; Descemet's membrane; and the endothelium. Behind the cornea is an aqueous-containing space called the anterior chamber. Importantly, the anterior chamber exerts pressure on the cornea to cause bio-mechanical consequences. Specifically, the anterior chamber of the eye exerts an intraocular pressure against the cornea. This creates stresses and strains that place the cornea under tension.

A typical cornea has a thickness of approximately 500 μm. From a bio-mechanical perspective, Bowman's capsule and the stroma are the most important layers of the cornea. Within the cornea, Bowman's capsule is a relatively thin layer (approximately 10 μm) that is located below the epithelium, within the anterior one hundred microns of the cornea. The stroma then comprises almost all of the remaining four hundred microns in the cornea. Further, the tissue of Bowman's capsule creates a relatively strong, elastic membrane that effectively resists forces in tension. On the other hand, the stroma comprises relatively weak connective tissue.

Bio-mechanically, Bowman's capsule and the stroma are both significantly influenced by the intraocular pressure that is exerted against the cornea by the anterior chamber. In particular, this pressure is transferred from the anterior chamber, and through the stroma, to Bowman's membrane. It is known that how these forces are transmitted through the stroma will affect the shape of the cornea. Thus, by disrupting forces between interconnective tissue in the stroma, the overall force distribution in the cornea can be altered. Consequently, this altered force distribution will then act against Bowman's capsule. In response, the shape of Bowman's capsule is changed, and due to the elasticity and strength of Bowman's capsule, this change will directly influence the shape of the cornea. With this in mind, and as intended for the present invention, refractive surgery is accomplished by making cuts on predetermined surfaces in the stroma to induce a redistribution of bio-mechanical forces that will reshape the cornea.

In view of the complexity of the cornea's internal structure and its related biomechanical properties, it is not surprising that surgical procedures alone may not resolve every optical aberration. In addition, surgical procedures may correct one eye problem while causing post-op optical aberrations that were not present before surgery. However, with the knowledge of the stromal tissue's biomechanical properties, the behavior of the stromal tissue in response to a laser procedure may be predicted. Thus, additional corrective measures may be developed based on the predicted behavior of the stromal tissue. One type of corrective measure that can be employed is a conventional correction means, such as spectacles or contact lenses. Spectacles or contact lenses can be selected or manufactured to compensate for pre-op or post-op corneal aberrations based on the needs of the patient.

In light of the above, it is an object of the present invention to provide a system and method for correcting a vision deficiency related to the depth-of-field of an eye. Another object of the present invention is to provide a two-step ophthalmic method for correcting a vision deficiency that combines a laser procedure with conventional vision correction. Yet another object of the present invention is to provide a vision correction method that is easy to use, is relatively simple to manufacture, and is relatively cost effective.

### SUMMARY OF THE INVENTION

In accordance with the present invention, a system for reshaping the cornea of an eye of a patient in order to increase the depth-of-field of the eye is used in a two-stage process. First, the depth-of-field is corrected with an intrastromal laser treatment. Preferably, this is done as disclosed in U.S. application Ser. No. 11/958,202, filed on Dec. 17, 2007, for an invention entitled “Method for Intrastromal Refractive Surgery” which is assigned to the same assignee as the present invention. Second, after the laser treatment has been completed, conventional vision correction is provided by spectacles or contact lenses. This conventional correction will then compensate for remaining pre-op aberrations, as well as residual aberrations in the patient's post-op vision (e.g. spherical, cylindrical, or sphero-cylindrical aberrations) that may have been induced during the laser treatment. As envisioned for the present invention, a manufacture of the conventional vision correction means for post-op vision will depend on preview testing that is conducted before the laser treatment is performed. As a consequence, the purpose of the present invention is to increase a patient's depth-of-field (i.e. laser treatment), correct for remaining pre-op aberrations, and compensate for induced aberrations (i.e. conventional vision correction means). Note: depending on the patient's tolerance for the laser treatment, there may be no induced aberrations to compensate for. If so, only pre-op aberrations need to be corrected by the conventional vision correction means.

Structurally, the system incorporates a diagnostic unit that includes a computer. One use of this diagnostic unit is for measuring a refractive error of the eye. Specifically, this is done to obtain a pre-op prescription for the eye that will correct the refractive error. Then, based on the measured refractive error and other properties of the eye, the laser treatment that will be used to increase the depth-of-field of the eye is planned. As implied above, a laser unit is provided in the system for use in performing the laser treatment. Preferably, this laser unit is a so-called femtosecond laser, but it can also be a picosecond or a nanosecond laser. Also, the computer is preferably programmed to take diagnostic data, such as the refractive error and other properties of the eye, as input, and to predict an induced refractive shift that will likely result from the laser treatment.

Based on the pre-op prescription and the predicted refractive shift, a preview lens is selected or manufactured for the system of the present invention. This preview lens can then be used to conduct a preview test on the eye of the patient. In particular, the purpose of this preview test is to determine the patient's toleration of the laser treatment. Specifically, the determination is made whether the post-op vision of the patient will tolerate a combination of the pre-op prescription and the predicted refractive shift.

An important aspect of the present invention is the use of a conventional vision correction means, after the laser treatment has been completed. In accordance with the present invention, this conventional vision correction means may be either spectacles or contact lenses but can also be intraocular lenses or other means of vision correction. In either case, the lenses are selected or manufactured according to the toleration of the patient, as determined in the preview test. For instance, when the preview test indicates the patient is intolerant, the vision correction lens is selected or manufactured to correct both the pre-op prescription and the refractive shift. On the other hand, when the preview test indicates patient toleration, the vision correction lens is made to correct only the pre-op prescription. It may happen, however, that after the laser treatment a patient may be emmetropic. If so, no further vision correction need be provided.

For the methods of the present invention, the first step involves measuring a refractive error for the eye of the patient. As mentioned above, this is done to obtain a pre-op prescription for correcting the refractive error. Next, a laser treatment is planned that will increase the depth-of-field of the eye. As part of this planning, a computer is programmed to calculate an induced refractive shift (for most patients, this is an unwanted side effect) that will likely result from the laser treatment that causes the induced refractive shift. After the pre-op prescription and the induced refractive shift have been identified, they are combined to create a preview plan that will be used for manufacturing the preview lens.

As noted above, the preview lens is selected or manufactured to evaluate the patient's tolerance for a predicted post-op vision. Once the patient's toleration has been established, the laser treatment to increase the depth-of-field of the eye is performed. After the laser treatment has been performed, it is then determined whether conventional correction means need to be employed to achieve good vision quality and comfort for the patient.

Based on the preview test, there are essentially three different conclusions for the method(s) of the present invention. First, when the patient is intolerant of the post-op vision, as determined in the preview test, a conventional correction means is provided that includes both the pre-op prescription and a correction for the refractive shift. Second, when the patient is tolerant of the post-op vision, as determined in the preview test, a conventional correction means is provided for correction of the pre-op prescription only. And, third, when a patient is emmetropic, no further action is required and no conventional correction means need be provided.

### BRIEF DESCRIPTION OF THE DRAWINGS

FIG. 1 is a schematic layout of the components of a system for reshaping the cornea with laser surgery, and for using conventional vision correction means to compensate for residual aberrations in accordance with the present invention; and

FIG. 2 is a functional flow chart of the methods performed for the present invention.

### DESCRIPTION OF THE PREFERRED EMBODIMENTS

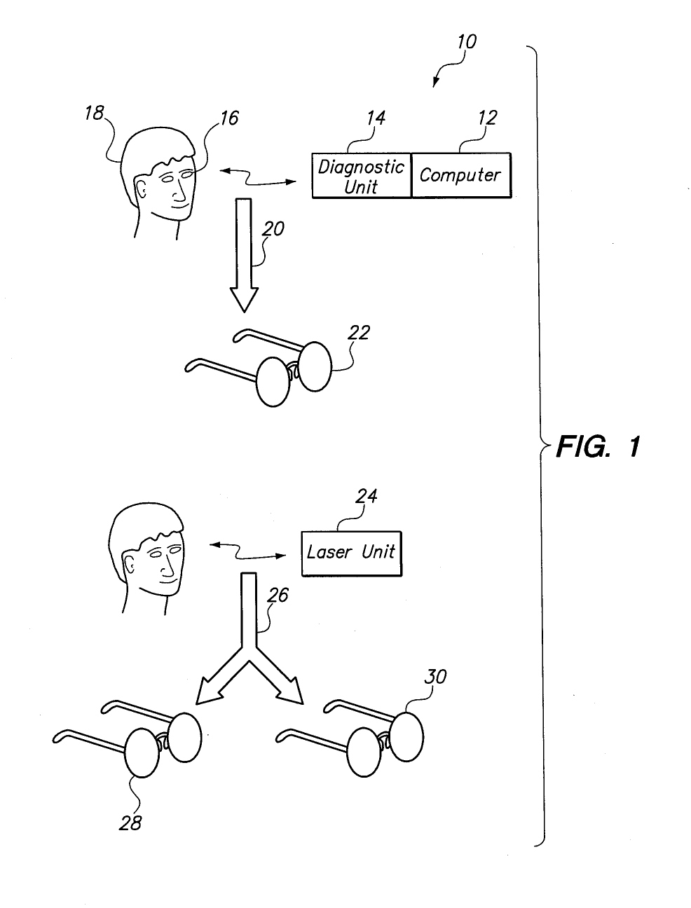

Referring initially to FIG. 1 the system of the present invention is shown and generally designated 10. As shown, a computer 12 is connected to a diagnostic unit 14. As envisioned for the present invention, the diagnostic unit 14 measures the refractive error of an eye 16 of a patient 18. In doing so, a pre-op prescription for the eye 16 is obtained. In addition to developing a pre-op prescription, the computer 12 and the diagnostic unit 14 are also used to predict the likely refractive shift in the cornea of the eye 16 that may be induced after a laser treatment is carried out.

Still referring to FIG. 1, once the computer 12 and diagnostic unit 14 develop a pre-op prescription and a predicted refractive shift (as shown by arrow 20), a preview lens 22 is created. The preview lens 22 is created to conduct a preview test to determine how the eye 16 of the patient 18 will react to the laser treatment. After the preview test is completed using the preview lens 22, the eye 16 of the patient 18 undergoes an instrastromal laser treatment using a laser unit 24. Once the laser unit 24 completes the laser treatment, a decision is made as indicated by decision point 26. At the decision point 26, either a first vision correcting lens 28 or a second vision correcting lens 30 is selected for use by the patient 18. The selection of either the first vision correcting lens 28 or the second vision correcting lens 30 is made according to whether the patient has tolerated the induced refractive shift caused by the laser unit 24. If the patient 18 has tolerated the induced refractive shift, the first vision correcting lens 28 is selected and conforms to the pre-op prescription calculated by the computer 12 and diagnostic unit 14. On the other hand, the second vision correcting lens 30 combines the pre-op prescription of the patient 18 and the actual induced refractive shift as measured after the laser treatment. In the case of an emmetropic patient, neither the first vision correcting lens 28 nor the second vision correcting lens 30 is required.

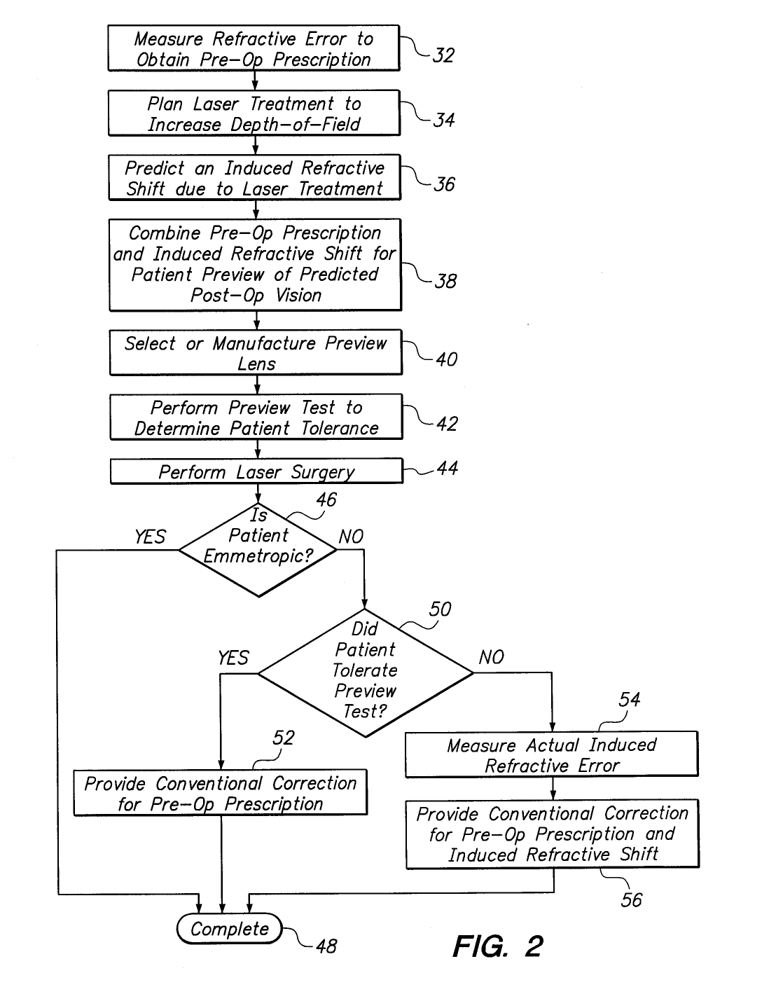

Now referring to FIG. 2, the steps required for the present invention are shown. First, the computer 12 and diagnostic unit 14 are used to measure the refractive error to obtain a pre-op prescription as indicated in action block 32. Next, a laser treatment using the laser unit 24 is planned to increase the depth of field of the eye 16 as indicated in action block 34. Then, as depicted in action block 36, the computer 12 will use data from the diagnostic unit 14 to predict an induced refractive shift due to the laser treatment plan discussed in the previous step. Following this prediction of an induced refractive shift, the computer 12 will combine the pre-op prescription and the predicted induced refractive shift as illustrated in action block 38. Once this step is completed, a preview lens 22 is selected or manufactured in accordance with the data supplied by the computer 12 and depicted in action block 40. As shown in action block 42, the preview lens 22 is then used to perform a preview test to determine the tolerance of the eye 16 of the patient 18 to the induced refractive shift caused by the laser treatment.

Still referring to FIG. 2, once the above-mentioned preparatory steps (action blocks 32-42) are conducted, the laser treatment is performed using the laser unit 24 and illustrated in action block 44. At this point, as shown in decision block 46, a determination is made as to whether the patient is emmetropic. If the patient is emmetropic, the procedure is complete as indicated in action block 48. If the patient is not emmetropic, it must be determined whether the eye 16 of the patient 18 tolerated the preview test as seen in decision block 50. If the answer is yes and the patient did tolerate the preview test, then the patient 18 is provided with the first conventional corrective lens 28 based only on the pre-op prescription and shown in action block 52. The procedure is then complete as shown in action block 48 for the patient 18 whose eye 16 tolerated the preview test using the preview lens 22. For the patient 18 whose eye 16 did not tolerate the preview test, measurements will have to be taken to measure the actual induced error as can be seen in action block 54. Based on these measurements, a conventional correction with a second vision correcting lens 30 is provided to the patient as demonstrated in action block 56. As stated previously, the second vision correcting lens 30 will compensate for the pre-op prescription and the induced refractive shift caused by the laser unit 24. Once either the first correction lens 28 or the second correction lens 30 is provided to the patient 18, the procedure for the non-emmetropic patient 18 is complete as shown in action block 48.

While the particular Method for Complementing Conventional Vision Correction with Laser Correction of the Cornea as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for reshaping the cornea of an eye of a patient to increase the depth-of-field of the eye, with subsequent compensation for induced residual aberrations, the system comprising:
a diagnostic unit for measuring a refractive error of the eye to obtain a pre-op prescription for the eye, and for using the pre-op prescription along with a prediction for an induced refractive shift resulting from laser treatment performed to increase the depth-of-field of the eye;
a preview lens for use in a preview test to determine a toleration of the patient for a combination of the pre-op prescription and the predicted refractive shift;
a laser unit for performing the laser treatment; and
a conventional vision correction means selected according to the toleration of the patient as determined in the preview test, wherein the selection is made from a group comprising a first vision correction lens for correction in accordance with only the pre-op prescription, and a second vision correction lens for correction in accordance with both the pre-op prescription and the refractive shift containing the residual aberrations.

2. A system as recited in claim 1 wherein the diagnostic unit further comprises a computer means for preparing the pre-op prescription, and for identifying the predicted refractive shift.

3. A system as recited in claim 2 wherein the computer means is in communication with the laser unit for control of the laser unit during the laser treatment, and is in communication with the diagnostic unit to determine efficacy of the preview test.

4. A system as recited in claim 1 wherein the first vision correction lens is selected from a group comprising a monocle, spectacles, intraocular lenses and contact lenses.

5. A system as recited in claim 1 wherein the second vision correction lens is selected from a group comprising spectacles, intraocular lenses and contact lenses.

6. A system as recited in claim 1 wherein the predicted refractive shift is provided as an input to the diagnostic unit.

7. A method for reshaping the cornea of an eye of a patient to increase the depth-of-field of the eye, with subsequent compensation for induced residual aberrations, the method comprising the steps of:
manufacturing a preview lens for use in a preview test of the patient to evaluate the tolerance of the patient to a predicted post-op vision;
performing laser surgery to increase the depth-of-field of the eye; and
providing a conventional vision correction means for eliminating induced aberration from actual post-op vision on the patient in accordance with patient tolerance determined in the preview test.

8. A method as recited in claim 7 further comprising the steps of:
measuring a refractive error for the eye of the patient to obtain a pre-op prescription for correcting the refractive error;
planning a laser treatment to increase the depth-of-field of the eye;
predicting an induced refractive shift due to the laser treatment; and
combining the pre-op prescription and the induced refractive shift to create a preview plan for use in manufacturing the preview lens.

9. A method as recited in claim 8 wherein the providing step is accomplished by creating the conventional correction means to include both the pre-op prescription and correction for the refractive shift when the patient is intolerant of the post-op vision as determined in the preview testing.

10. A method as recited in claim 8 wherein the providing step is accomplished by creating the conventional correction means for correction of the pre-op prescription when the patient is tolerant of the post-op vision as determined in the preview testing.

11. A method as recited in claim 7 wherein the providing step is accomplished by taking no further action when the patient is emmetropic after the performing step.

12. A method for determining a post-op compensation for an eye of a patient due to residual aberrations induced during a laser treatment, the method comprising the steps of:
measuring a refractive error for the eye of the patient to obtain a pre-op prescription for correcting the refractive error;
planning a laser treatment to increase the depth-of-field of the eye;
predicting an induced refractive shift due to the laser treatment;
combining the pre-op prescription and the induced refractive shift to create a preview plan for use in manufacturing a preview lens; and
testing the eye with the preview lens to determine a need for a conventional vision correction means after the laser treatment.

13. A method as recited in claim 12 wherein the testing step determines whether the patient is tolerant to post-op vision.

14. A method as recited in claim 13 further comprising the step of creating a conventional correction means to include both the pre-op prescription and correction for the refractive shift when the patient is intolerant of the post-op vision.

15. A method as recited in claim 13 further comprising the step of creating a conventional correction means for correction of the pre-op prescription when the patient is tolerant of the post-op vision.

16. A system for reshaping the cornea of an eye of a patient to increase the depth-of-field of the eye, with subsequent compensation for induced residual aberrations, the system comprising:
a means for measuring a refractive error for the eye of the patient to obtain a pre-op prescription for correcting the refractive error;
a means for predicting an induced refractive shift due to the laser treatment;
a means for creating a preview plan for use in manufacturing a preview lens;
a means for testing the eye with the preview lens to determine a need for a conventional vision correction after the laser treatment;
a means for performing the laser treatment; and
a means for conventional vision correction.

17. A system as recited in claim 16 wherein the means for measuring a refractive error and the means for predicting an induced refractive shift are both accomplished using a diagnostic unit.

18. A system as recited in claim 16 wherein the means for conventional vision correction is selected from a group comprising a first vision correction lens and a second vision correction lens.

19. A system as recited in claim 18 wherein the first vision correction lens is selected for correction in accordance with only the pre-op prescription.

20. A system as recited in claim 18 wherein the second vision correction lens is selected for correction in accordance with both the pre-op prescription and the induced refractive shift due to laser treatment.
